(19) United States
(12) Patent Application Publication
(10) Pub. No.: US 2023/0190171 A1
(43) Pub. Date: Jun. 22, 2023

(54) ELECTROPHYSIOLOGICAL (EP) MAP POINTS ADJUSTMENTS BASED ON USER CLINICAL INTERPRETATION

(71) Applicant: BIOSENSE WEBSTER (ISRAEL) LTD., Yokneam (IL)

(72) Inventors: Fady Massarwa, Baka Al Gharbiyya (IL); Meytal Segev, Haifa (IL); Sigal Altman, Ramat Hashofet (IL)

(21) Appl. No.: 17/555,629

(22) Filed: Dec. 20, 2021

(51) Int. Cl. A61B 5/367; A61B 5/00

(52) U.S. Cl. CPC A61B 5/367; A61B 5/7475; A61B 5/6852

(57) ABSTRACT

In one example, method includes receiving an electrophysiological (EP) map of at least a portion of a surface of a cardiac chamber, the EP map including multiple EP values overlayed at multiple respective positions on the surface. A clinical input is identified, that was marked on the EP map by a user using an input device. One or more of the EP values are automatically adjusted to be consistent with the clinical input.

### FIELD OF THE DISCLOSURE

The present disclosure relates generally to electrophysiological mapping, and particularly to manually-assisted editing of cardiac electrophysiological maps.

### BACKGROUND OF THE DISCLOSURE

Editing tools for assisting in the interpretation of an electrophysiological map were previously proposed in the patent literature. For example, U.S. Pat. No. 8,478,393 describes a method for visualization of electrophysiology data representing electrical activity on a surface of an organ over a time period. An interval within the time period is selected in response to a user selection. Responsive to the user selection of the interval, a visual representation of physiological information for the user selected interval is generated by applying at least one method to the data. The visual representation is spatially represented on a graphical representation of a predetermined region of the surface of the organ.

### BRIEF DESCRIPTION OF THE DRAWINGS

The present disclosure will be more fully understood from the following detailed description of the examples thereof, taken together with the drawings in which:

FIG. 1 is a schematic, pictorial illustration of a system for electrophysiological (EP) mapping, in accordance with an example of the present disclosure;

FIGS. 2A-2C are schematic, pictorial EP maps overlayed with user clinical inputs of propagation paths (2A and 2B) and region marking (2C), in accordance with examples of the present disclosure;

FIG. 3 is a schematic drawing of a hinted activation path provided by a user as clinical input, along with annotation times recalculated to be consistent with the hinted path, in accordance with an example of the present disclosure;

FIGS. 4A-4C are schematic illustrations of steps in the recalculation of annotation times shown in FIG. 3, to be consistent with the hinted path, in accordance with an example of the present disclosure; and

FIG. 5 is a flow chart that schematically illustrates a method for adjustment of data points of an EP map using user clinical input, in accordance with another example of the present disclosure.

### DETAILED DESCRIPTION OF EXAMPLES

### Overview

Catheter-based electrophysiological (EP) mapping techniques may produce various types of EP maps of an organ, such as a left atrium of a heart. Cardiac EP maps, such as a local activation time (LAT) map, a bipolar potential map, or a unipolar potential map, are produced by acquiring electrograms from locations on a heart chamber surface. EP values, such as LATs (or potentials), are then derived from the electrograms for the locations. Such locations and respective EP values, called hereafter “data points,” are then overlayed, typically using colors, onto a 3D map of the chamber.

In practice, analysis of the vast number of data points that are acquired may lead to erroneous results in an EP map. Errors in EP maps are due to various difficulties, such as during acquisition (e.g., low signal to noise ratio, mechanical distortion of a cardiac wall by a catheter), and in the analysis stages (e.g., erroneous time annotations of activations).

For example, some of the LAT annotations may become inaccurate in complicated algorithms. To mitigate inaccuracies, an LAT consistency algorithm may be used to identify inaccurate LATs, and, once identified, an inaccurate LAT is not used to color the map. LAT corrections may be based on altering a window of interest (WOI—a portion of the cardiac cycle used for LAT estimation) over the EP signal and/or adjusting a threshold in the LAT consistency algorithm. Still, none of the above methods prevent incorporating outlier EP values into an EP map.

Therefore, typically, the physician often corrects inaccuracies manually when observed. Such manual correction by the physician is tedious and time consuming. Moreover, as the number of acquired data points increases with modern multi-electrode catheters, it becomes increasingly impractical for a physician to perform manual correction.

Examples of the present disclosure that are described hereinafter provide methods and systems that utilize clinical input provided by the physician to improve the accuracy in a specific portion of an EP map. For example, clinical input from an experienced physician can be used to automate the corrections that would typically be made manually by the same physician. Rather than having the physician enter pinpointed manual corrections, the disclosed technique relies instead on high-level insights made by the physician, typically by letting the physician enter certain general clinically meaningful tendencies on the EP map (e.g., draw on a touchscreen displaying the map), and then automatically adjusting the map so that the points are consistent with these observed “global” tendencies.

In one example, the physician may draw a directional curve showing a clinically observed direction of EP wave propagation. The automatically calculated LATs along that arrow may be recomputed to provide more accurate LATs based on this additional input. The location and direction of the arrow may be used by the disclosed technique and algorithm to improve the LAT map in areas that are clinically critical.

Improved accuracy may be attained, for example, by moving the WOI or by defining a smaller WOI for determining LAT. The new WOI may be determined based on the direction of the arrow provided by the physician as well as neighboring LATs. The direction of the arrow may also provide input to the LAT consistency algorithm so that the clinical input is considered when selecting outliers.

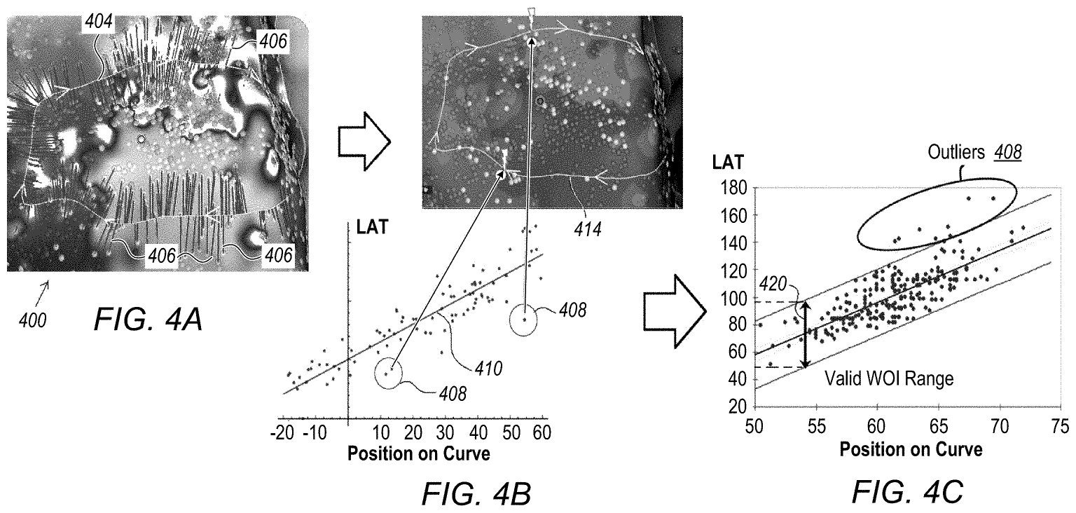

In an example, a processor receives an EP map with user clinical input in a form of hint-activation paths. The processor sorts the data points (each data point made of an EP value at a position on the EP map, as shown in FIG. 4A). Then, the processor segments the path by running piecewise regression to find a best fitting LAT activation path. The processor adjusts WOI for each data point and uses the adjusted WOI recomputed annotations (e.g., LAT annotations) to generate a more accurate EP map and, importantly, one that is consistent with the clinical input.

In another example, a physician may circle an area that is clinically observed to have a specific characteristic. This clinical input may be used to improve the mapping. For example, the sensitivity of the LAT consistency algorithm may be adjusted based on the classification of the region. In one example, the characteristic is scarred tissue. In this case, points inside the circled area may not be classified as outliers. By automating EP map editing based on user insights that are given in the form of informal drawings on the map, the technique improves map quality of multi-electrode catheter systems that acquire a vast number of data points in a short period of time.

Typically, the processor is programmed in software containing a particular algorithm that enables the processor to conduct each of the processor-related steps and functions outlined above.

By increasing EP map accuracy using the aforementioned interactive graphical means provided to the physician, and an algorithm to implement a physician's informal (e.g., hand drawn) inputs, the disclosed techniques may assist the physician in the interpretation of EP maps and thus expedite and improve the quality of complicated diagnostic tasks, such as those required in diagnostic catheterizations.

### System Description

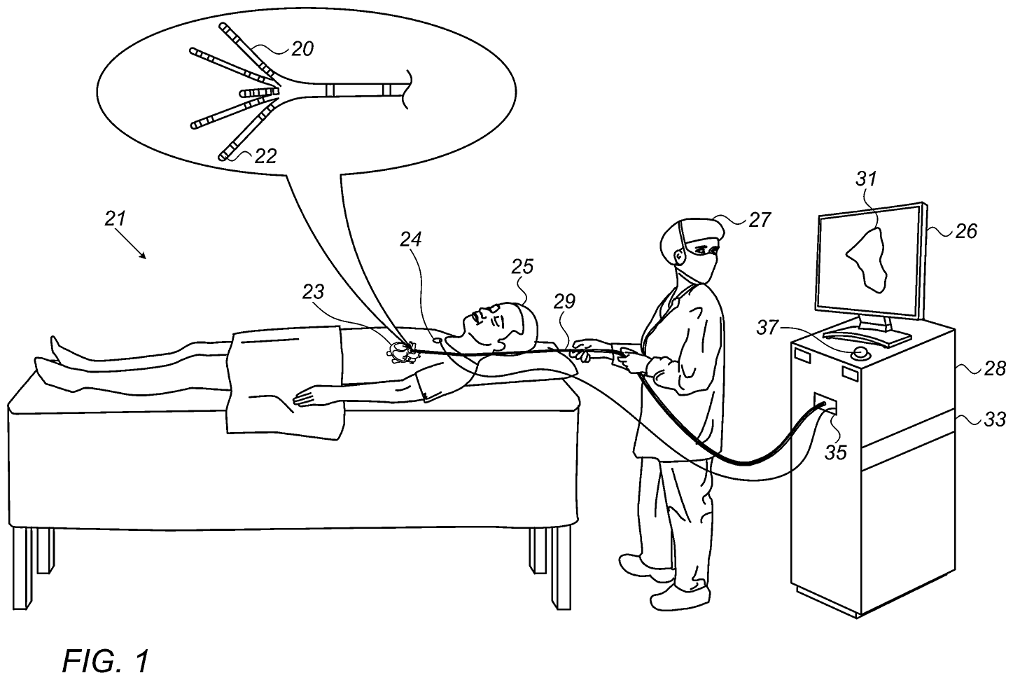

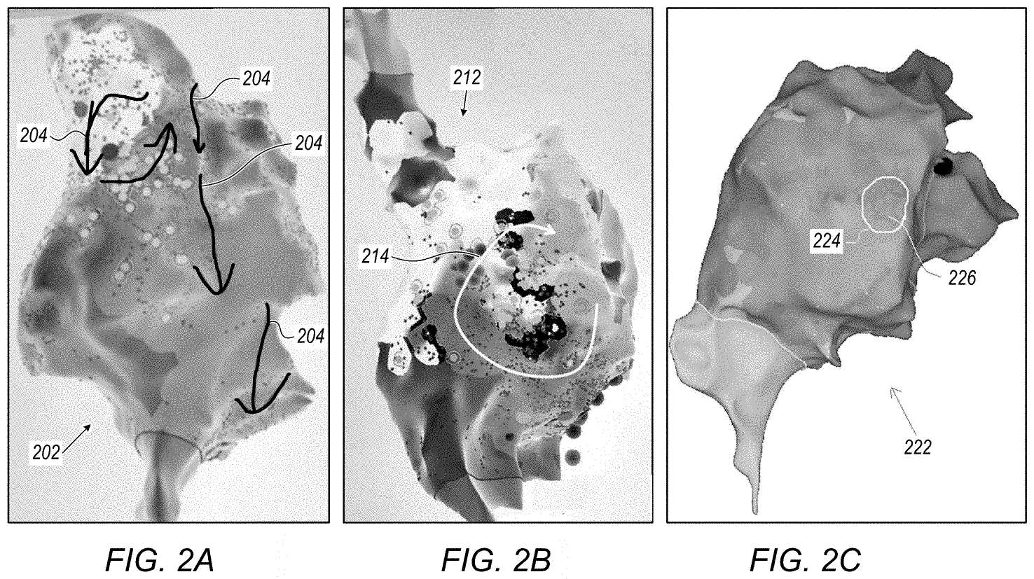

FIG. 1 is a schematic, pictorial illustration of a system 21 for electrophysiological (EP) mapping, in accordance with an example of the present disclosure. FIG. 1 depicts a physician 27 using a mapping Pentaray® catheter 29 to perform an EP mapping of a heart 23 of a patient 25. Catheter 29 comprises, at its distal end, one or more arms 20, which may be mechanically flexible, each of which is coupled with one or more electrodes 22. During the mapping procedure, electrodes 22 acquire and/or inject unipolar and/or bipolar signals from and/or to the tissue of heart 23. A processor 28 receives these signals via an electrical interface 35, and uses information contained in these signals to construct an EP map 31 stored by processor 28 in a memory 33. During and/or following the procedure, processor 28 may display EP map 31 on a display 26, wherein display 26 can be a touchscreen to enable physician 27 marking clinical inputs on EP map 31, such as marking activation paths and scar regions, as shown in FIGS. 2A-2C. Alternatively or additionally, physician 27 may mark the clinical inputs using any other suitable input device, e.g., in the form of a mouse or a trackball 37.

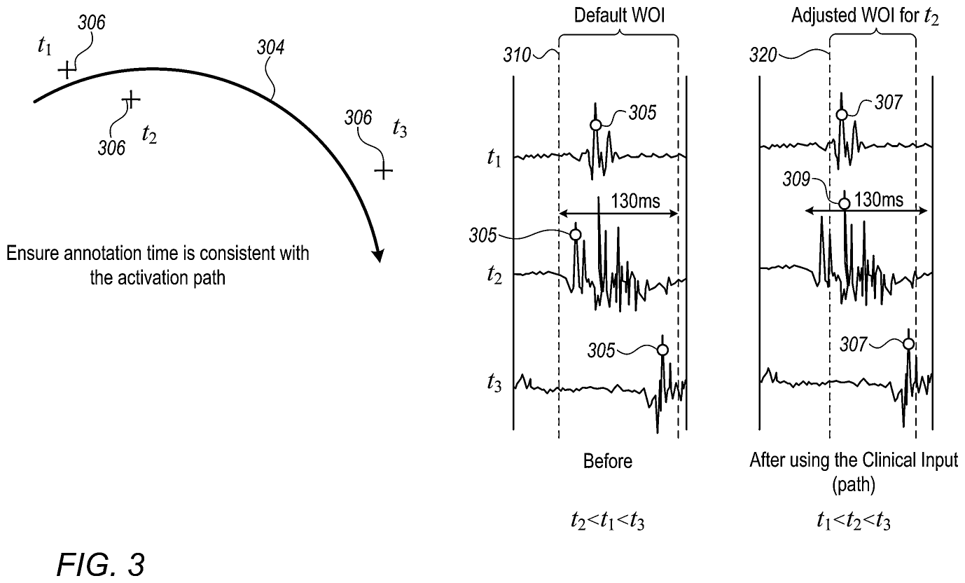

EP map 31 may be an LAT map, a bipolar potential map, or another map type. The quality of EP map 31 is improved by using the disclosed technique to derive and present a confidence level on the map, as described in FIG. 2 and FIG. 3.

During the procedure, a tracking system is used to track the respective locations of sensing electrodes 22, such that each of the signals may be associated with the location at which the signal was acquired. For example, the Active Catheter Location (ACL) system, made by Biosense-Webster (Irvine Calif.), which is described in U.S. Pat. No. 8,456,182, whose disclosure is incorporated herein by reference, may be used. In the ACL system, a processor estimates the respective locations of the electrodes based on impedances measured between each of the sensing-electrodes 22, and a plurality of surface electrodes 24 that are coupled to the skin of patient 25. For example, three surface electrodes 24 may be coupled to the patient's chest and another three surface electrodes may be coupled to the patient's back. For ease of illustration, only one surface electrode is shown in FIG. 1. Electric currents are passed between electrodes 22 inside heart 23 of the patient and surface-electrodes 24. Processor 28 calculates an estimated location of all electrodes 22 within the patient's heart based on the ratios between the resulting current amplitudes measured at surface electrodes 24 (or between the impedances implied by these amplitudes) and the known positions of electrodes 24 on the patient's body. The processor may thus associate any given impedance signal received from electrodes 22 with the location at which the signal was acquired.

The example illustration shown in FIG. 1 is chosen purely for the sake of conceptual clarity. Other tracking methods can be used, such as ones based on measuring voltage signals. Other types of sensing catheters, such as the Lasso® Catheter (produced by Biosense Webster) or basket catheters may equivalently be employed. Physical contact sensors may be fitted at the distal end of mapping catheter 29 to estimate contact quality between each of the electrodes 22 and an inner surface of the cardiac chamber during measurement.

Processor 28 typically comprises a general-purpose computer with software programmed to carry out the functions described herein. In particular, processor 28 runs a dedicated algorithm as disclosed herein, including in FIG. 3, that enables processor 28 to perform the disclosed steps, as further described below. The software may be downloaded to the computer in electronic form, over a network, for example, or it may, alternatively or additionally, be provided and/or stored on non-transitory tangible media, such as magnetic, optical, or electronic memory.

### User Clinical Input Marked an Ep Map

As noted above, some acquired point attributes, such as LAT values and filtering status (LAT consistency), are determined by mathematical algorithms that, in many cases, do not take the clinical diagnosis and observations of the physician into account. For example, in some arrhythmias the automatic computation of LAT values of the point in the reentry path are inaccurate, which may lead to misleading coloring and consistency determinations.

Typically, the user manually iterates over each one of the problematic points and fixes them manually (for example, by fixing the annotation or changing the consistency outlier classification). This process is tedious and, in case of multiple points, the user may not find them all.

Utilizing clinical physician hints, such as a general wave-propagation direction in specific areas, can be useful to automate this process and help the algorithm obtain better results. This disclosure describes how to incorporate various physician guidelines/hints that are based on a clinical understanding of the study into point-related algorithms, such as LAT consistency and map annotation algorithms.

The disclosure considers two types of clinical hints:
1. Directional. In this case, the physician outlines one or more directed curves on the map surface that should provide a hint about wave propagation direction or a line of blocks (based on user clinical observation). Using these curves, the map annotation algorithm can be improved by having a tighter WOI (or possibly a fixed WOI position) for each point which is determined by its nearest location on the curve (path). The new WOI is calculated based on the hint and the neighboring point annotation. Additionally, the directional curves can serve as an input for the current LAT consistency algorithm and thus improve the outlier decision for each point by considering clinical values and not only statistical regional values. Specifically, these curves can help to build a more reliable conduction path that is used in the second stage of an LAT consistency algorithm.
2. Regional. In this case, the clinical hint is over some area on the map surface which can be interpreted in several ways that can help classify the underlying points in this area, such as: (2a) a scar area where all the points inside are considered as a scar, and (2b) a high-level certainty area where points inside are never classified as outliers.

FIGS. 2A-2C are schematic, pictorial EP maps overlayed with user clinical inputs of propagation paths (204 and 214 on FIGS. 2A and 2B, respectively) and region 226 marking (224 on FIG. 2C), in accordance with examples of the present disclosure.

In EP map 202 of FIG. 2A (an LAT map), a physician drew hinted activation paths 204 as a clinical input, with the expectation that the LAT values would be consistent along those paths (e.g., monotonically increasing).

Similarly, in EP map 212 of FIG. 2B, which is also an LAT map, a physician drew a hinted activation path 214 as a clinical input, with the expectation that the LAT values would also be consistent along this semicircular path (e.g., monotonically increasing).

In EP map 222 of FIG. 2C, which can be an LAT map or a potential map, a physician drew closed curve 224 to mark a hinted region 226 as a clinical input, with the expectation that region 226 is on the map surface, and may be interpreted in several ways that can help classify the underlying points in this area, such as:
Scar area 226, where all included points are considered as a scar
High level certainty area 226, where included data points are never classified as outliers

### Using User Clinical Input of Hinted Activation Paths to Improve an Lat Map

FIG. 3 is a schematic drawing of a hinted activation path 304 provided by a user as clinical input along with annotation times (307, 309) recalculated to be consistent with the hinted path, in accordance with an example of the present disclosure.

As seen, there are three data points 306 in the vicinity of hinted activation path 304, where data points 306 are activation times t₁, t₂ and t₃ at respective locations over an LAT map, such as maps 202 and 212 of FIG. 2. The automatically calculated annotations 305 on the three respective electrograms, within a default WOI 310 (e.g., of time duration of 130 mSec), are inconsistent with the hinted activation path, since times are not monotonically increasing along with t₁>t₂. Using the hinted activation path, the disclosed technique adjusts default WOI 310 into shorter WOI 320 (e.g., with time duration smaller than 130 mSec of WOI 310) and recalculates the annotations times.

As seen, two annotation times 307 are unchanged by the change in WOI. However, the erroneous annotation time 305 is correctly found by the algorithm to be annotation 309, which is consistent with the hinted activation path, as times are now monotonically increasing along the path, with t₁<t₂.

### Algorithm for Improving an Lat Map Using User Clinical Input of Hinted Activation Paths

FIGS. 4A-4C are schematic illustrations of steps in the recalculation of annotations times, as shown in FIG. 3, to be consistent with the hinted path, in accordance with an example of the present disclosure.

FIG. 4A shows a hinted activation path 404 provided by a user as clinical input. Path 404 is drawn on an LAT map 400, and is generally similar to path 214 of FIG. 2B. As seen, the algorithm has used certain criteria to select (406) EP values relevant to reconsideration (e.g., recalculation of activation times) based on the hinted path. These selected points are in the vicinity of the curve (along the curve) with their distance from the hinted curve being smaller than a threshold that is defined as one of the algorithm parameters (this threshold can be preset or calculated automatically according to available data points). The selected data points are then projected to the curve and sorted according to their position in the curve (the closest to the curve beginning is the earlier in time).

As can be expected, path 404 is crude and, as shown in FIG. 4B, the algorithm uses a regression model to generate (e.g., segment (410)) an adjusted activation path (414) that is more accurate than the hinted path 404, due to path 414 being based on LAT data points selected (406) for reconsideration, though the adjustment statistically neglects outlier values 408.

The result, seen in FIG. 4C, is that for each position on curve 404, the algorithm provides a valid WOI 420, and the annotation times of the reconsidered data points are calculated (as shown in FIG. 3) based on the valid WOI, which are used to generate an EP map consistent with the user's clinical input, as annotation times (307, 309) are recalculated in FIG. 3.

### Method for Improving an Ep Map Using User Clinical Input of Hinted Activation Paths

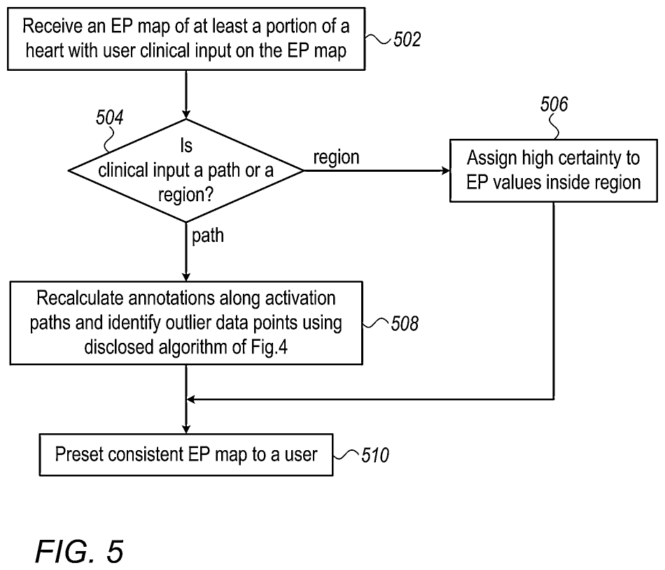

FIG. 5 is a flow chart that schematically illustrates a method for adjusting data points of an EP map using user clinical input, in accordance with another example of the present disclosure. The algorithm, according to the presented example, carries out a process that begins with processor 28 receiving an EP map (e.g., an LAT map) of at least a portion of a heart, with user clinical input on the map (e.g., a drawn hinted activation path), at a clinical input receiving step 502.

Next, the processor checks the clinical input type, if it is, for example, an activation path and/or a closed region, at a clinical input type checking step 504.

In case the clinical input is considered a region, an assigning step 506 may include an adjustment of the sensitivity of an LAT consistency algorithm based on the classification of the region. This may exclude data points inside the region from being classified as outliers. The classification is done using the WOI being centered around the regression line (at each point), and every point that is outside the updated WOI should be considered as an outlier (see FIG. 4C). In another example, all of the points inside such a region may be considered as a scar.

In case the clinical input considered is one or more hinted activation paths, step 508 may include performing the algorithm described in FIG. 4 to ensure EP map consistency along any hinted activation paths, and to identify outlier data points.

Finally, at an EP map presentation step 510, processor 28 presents the updated EP map after all clinical inputs were used in making the EP map more consistent with clinical observation by the physician. As described above, using the disclosed technique, the EP map correction is made by the physician who provides high level clinical inputs and without requiring meticulous, laborious, manual work on the side of the physician.

The example flow chart shown in FIG. 5 is chosen purely for the sake of conceptual clarity. In other examples, other types of clinical inputs may be considered, such as drawings that hint at several electro potential waves that collision at some point, or selecting a path with some thickness to indicate a scar region or slow conduction region.

### EXAMPLE 1

A method including receiving an electrophysiological (EP) map (310 of at least a portion of a surface of a cardiac chamber, the EP map comprising multiple EP values overlayed at multiple respective positions on the surface. A clinical input (204, 214, 224) is identified, that was marked on the EP map by a user using an input device (26, 37). One or more of the EP values are automatically adjusted to be consistent with the clinical input (204, 214, 224).

### EXAMPLE 2

The method according to claim 1, and comprising adjusting one or more of the positions to be consistent with the clinical input (204, 214, 226).

### EXAMPLE 3

The method according to claim 1, wherein the clinical input is indicative of one or more activation paths (204, 214).

### EXAMPLE 4

The method according to claim 3, wherein the one or more activation paths (204, 214) are electronically drawn on the EP map (31) using a touchscreen (26) displaying the EP map (31).

### EXAMPLE 5

The method according to claim 3, wherein adjusting the EP values comprises adjusting a window of interest (WOI) (310) on an electrogram and annotating (307, 309) the electrogram based on the adjusted WOI (320).

### EXAMPLE 6

The method according to claim 1, wherein the clinical input is indicative of one or more regions (226) on the EP map (31).

### EXAMPLE 7

The method according to claim 6, wherein the one or more regions (226) are electronically drawn on the EP map (31) using a touchscreen (26) displaying the EP map (31).

### EXAMPLE 8

The method according to claim 7, wherein adjusting the EP values comprises adjusting a level-of-confidence threshold of the EP values in the one or more regions (226).

### EXAMPLE 9

The method according to any of claims 1 through 8, wherein identifying the clinical input (204, 214, 226) comprises applying predefined inclusion criteria to determine which of the EP values is to be considered in relation with the identified clinical input (204, 214, 226).

### EXAMPLE 10

The method according to any of claims 1 through 9, wherein the EP values are one of local activation times (LATs), bipolar potentials, and unipolar potentials.

### EXAMPLE 11

The method according to any of claims 1 through 10, wherein the input device is one of a touchscreen (26), a computer mouse, and a trackball (37).

### EXAMPLE 12

A system comprising a memory (33) and a processor (28). The memory (33) is configured to store an electrophysiological (EP) map (31) of at least a portion of a surface of a cardiac chamber, the EP map (31) comprising multiple EP values overlayed at multiple respective positions on the surface. The processor (28) is configured to (i) receive a clinical input (204, 214, 226) marked on the EP map (31) by a user using an input device (26, 37), and (ii) automatically adjust one or more of the EP values to be consistent with the clinical input(204, 214, 226).

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A method for electrophysiological mapping, comprising:
receiving an electrophysiological (EP) map of at least a portion of a surface of a cardiac chamber, the EP map comprising multiple EP values overlayed at multiple respective positions on the surface;
identifying a clinical input marked on the EP map by a user using an input device; and
automatically adjusting one or more of the EP values to be consistent with the clinical input.

2. The method according to claim 1, and comprising adjusting one or more of the positions to be consistent with the clinical input.

3. The method according to claim 1, wherein the clinical input is indicative of one or more activation paths.

4. The method according to claim 3, wherein the one or more activation paths are electronically drawn on the EP map using a touchscreen displaying the EP map.

5. The method according to claim 3, wherein adjusting the EP values comprises adjusting a window of interest (WOI) on an electrogram and annotating the electrogram based on the adjusted WOI.

6. The method according to claim 1, wherein the clinical input is indicative of one or more regions on the EP map.

7. The method according to claim 6, wherein the one or more regions are electronically drawn on the EP map using a touchscreen displaying the EP map.

8. The method according to claim 7, wherein adjusting the EP values comprises adjusting a level-of-confidence threshold of the EP values in the one or more regions.

9. The method according to claim 1, wherein identifying the clinical input comprises applying predefined inclusion criteria to determine which of the EP values is to be considered in relation with the identified clinical input.

10. The method according to claim 1, wherein the EP values are one of local activation times (LATs), bipolar potentials, and unipolar potentials.

11. The method according to claim 1, wherein the input device is one of a touchscreen, a computer mouse, and a trackball.

12. A system for electrophysiological mapping, comprising:
a memory configured to store an electrophysiological (EP) map of at least a portion of a surface of a cardiac chamber, the EP map comprising multiple EP values overlayed at multiple respective positions on the surface; and
a processor, which is configured to:
receive a clinical input marked on the EP map by a user using an input device; and
automatically adjust one or more of the EP values to be consistent with the clinical input.

13. The system according to claim 12, wherein the processor is further configured to adjust one or more of the positions to be consistent with the clinical input.

14. The system according to claim 12, wherein the clinical input is indicative of one or more activation paths.

15. The system according to claim 14, wherein the one or more activation paths are electronically drawn on the EP map using a touchscreen displaying the EP map.

16. The system according to claim 14, wherein the processor is configured to adjust the EP values by adjusting a window of interest (WOI) on an electrogram and annotating the electrogram based on the adjusted WOI.

17. The system according to claim 12, wherein the clinical input is indicative of one or more regions on the EP map.

18. The system according to claim 17, wherein the one or more regions are electronically drawn on the EP map using a touchscreen displaying the EP map.

19. The system according to claim 18, wherein the processor is configured to adjust the EP values by adjusting a level-of-confidence threshold of the EP values in the one or more regions.

20. The system according to claim 12, wherein the processor is configured to identify the clinical input by applying predefined inclusion criteria to determine which of the EP values is to be considered in relation with the identified clinical input.

21. The system according to claim 12, wherein the EP values are one of local activation times (LATs), bipolar potentials, and unipolar potentials.

22. The system according to claim 12, wherein the input device is one of a touchscreen, a computer mouse, and a trackball.
